(19) United States
(12) Patent Application Publication
(10) Pub. No.: US 2024/0316044 A1
(43) Pub. Date: Sep. 26, 2024

(54) GRANULATE COMPOSITION COMPRISING NILOTINIB

(71) Applicant: HELM AG, Hamburg (DE)

(72) Inventors: Katja DREYER, Hamburg (DE); Gustavo Franco COSTA, Coimbra (PT); Gabriel Leitao SILVA, Lordemao Coimbra (PT)

(73) Assignee: HELM AG, Hamburg (DE)

(21) Appl. No.: 18/571,942

(22) PCT Filed: Jun. 15, 2022
(86) PCT No.: PCT/EP2022/066305

§ 371 Date: Dec. 19, 2023

(30) Foreign Application Priority Data
Jun. 19, 2021 (EP) 21180485.1

(51) Int. Cl. A61K 31/501; A61K 9/16

(52) U.S. Cl. CPC A61K 31/501; A61K 9/1611; A61K 9/1617; A61K 9/1635; A61K 9/1641; A61K 9/1682

(57) ABSTRACT

The invention relates to a pharmaceutical composition comprising (i) granules comprising Nilotinib, preferably in crystalline form, or a physiologically acceptable salt and/or solvate thereof, preferably crystalline anhydrous Nilotinib hydrochloride or crystalline Nilotinib hydrochloride monohydrate; and (ii) an extragranular phase comprising a surfactant, preferably poloxamer. The granules are preferably dry granulated.

Priority is claimed of European patent application no. 21 180 485.1 that was filed on Jun. 19, 2021.

The invention relates to a pharmaceutical composition comprising (i) granules comprising Nilotinib, preferably in crystalline form, or a physiologically acceptable salt and/or solvate thereof, preferably crystalline anhydrous Nilotinib hydrochloride or crystalline Nilotinib hydrochloride monohydrate; and (ii) an extragranular phase comprising a surfactant, preferably poloxamer. The granules are preferably dry granulated.

Nilotinib is commercialized under the tradename Tasigna® in form of hard gelatin capsules. According to the summary of product characteristics (SmPC), the capsules contain Nilotinib as hydrochloride monohydrate, lactose monohydrate, crospovidone type A, poloxamer 188, colloidal anhydrous silica, and magnesium stearate. The manufacturing process of Tasigna® is a standard process including aqueous wet granulation, drying, screening, mixing and encapsulation (https://www.ema.europa.eu/en/documents/scientific-discussion/tasigna-epar-scientific-discussion_en.pdf).

EP-A 3 501 505 relates to a crystalline dispersion of nilotinib hydrochloride in a polymeric matrix, its use for the preparation of a drug form and a preparation method of this dispersion using the hot melt extrusion method.

WO 2007/015870 relates to crystalline forms of Nilotinib free base and salts thereof are prepared by various processes including Nilotinib HCl form A and Nilotinib HCl form B.

WO 2008/037716 relates to a pharmaceutical composition, especially capsules, comprising granules containing Nilotinib or a salt thereof with at least one pharmaceutically acceptable excipient. The granules may be produced by a wet granulation process.

WO 2010/054056 relates to various crystalline forms of Nilotinib hydrochloride denoted as forms T1 to T19. Nilotinib HCl crystalline form T17 is anhydrous and does not convert to other forms when heated, while other polymorphs, known in the art, such as Nilotinib HCl form A and Nilotinib HCl form B are converted to other polymorphs.

WO 2012/164578 relates to pharmaceutical compositions comprising Nilotinib or a pharmaceutically acceptable salt thereof and a process for the preparation of the same. A process for preparing dry-granulated pharmaceutical composition of Nilotinib is disclosed comprising compacting nilotinib hydrochloride alone or mixing with one or more of pharmaceutically acceptable excipient(s) by roller compactor or slugging; sizing the compacts or slugs into granules by milling; and mixing the granules with one or more of pharmaceutically acceptable excipients to form the composition. In some of the examples of WO 2012/164578, poloxamer is used as an intragranular surfactant.

WO 2012/174082 relates to soluble pharmaceutical compositions of amorphous nilotinib or a pharmaceutically acceptable salt thereof using one or more organic acids that function as a solubilizing agent, increasing the bioavailability of nilotinib and suppressing the food effect associated with certain compositions of nilotinib. A spray dried mixture of Nilotinib and several excipients is subsequently mixed with poloxamer and then roller compacted such that poloxamer is present as an intragranular surfactant.

WO 2017/064538 relates to a composition comprising nilotinib hydrochloride anhydrous form T17, wherein crospovidone is used as filler in the formulation instead of prior art fillers like microcrystalline cellulose and lactose. In the examples of WO 2017/064538, poloxamer is used as an intragranular surfactant. Further, WO 2017/064538 reports the during formulation studies it was found that Nilotinib HCl crystalline form T17 is not compatible with most excipients used in pharmaceutical formulations. Especially, common diluents and fillers such as lactose anhydrous, lactose monohydrate, cellulosic excipients such as microcrystalline cellulose, mannitol used in prior art formulations (WO 2008/037716, WO 2012/164578, WO 2014/060449, WO 2014/174496) comprising Nilotinib have been observed to be incompatible with nilotinib anhydrous form T17. Hence the resulting formulation experiences high polymorphic conversion.

WO 2020/095187 relates to a crystalline form of nilotinib hydrochloride, process for its preparation and pharmaceutical composition comprising the same.

N. J. Koehl et al., Mol. Pharmaceutics 2020, 17, 9, 3202-3213, reports about an investigation exploring the impact of surfactant type and digestion and reveals that highly digestible surfactants improve oral bioavailability of Nilotinib. Medium-chain fatty acid-based surfactants are found to be more readily digested compared to long-chain surfactants, and for Nilotinib highly digestible, medium chain-based surfactants are preferred.

The known pharmaceutical compositions and dosage forms of Nilotinib are not satisfactory in every respect and there is a demand for alternative or improved pharmaceutical compositions and dosage forms of Nilotinib, especially of crystalline anhydrous Nilotinib hydrochloride or crystalline Nilotinib hydrochloride monohydrate.

It is an object of the invention to provided pharmaceutical compositions and dosage forms of Nilotinib that have advantages over the prior art.

This object has been achieved by the subject-matter of the patent claims.

It has been surprisingly found that pharmaceutical compositions and dosage forms can be prepared that are bioequivalent to commercial Tasigna® and that contain Nilotinib and surfactant in separate phases, namely Nilotinib in the intragranular phase and the surfactant in the extragranular phase.

Formulating poorly water-soluble drugs such as Nilotinib and surfactants such as poloxamer in separate phases (intragranular vs. extragranular) is unusual. One would typically expect that the surfactant can evolve its solubilizing effect only when it is intimately mixed with the drug (i.e. intragranularly), but not when it is formulated in a separate phase (i.e. extragranularly). The possible impact of the surfactant at particle surfaces in the microenvironment around the dissolving particles where local concentrations are high should be considered.

This is also the reason why e.g. in hydrophilization processes a solution of a hydrophilizing excipient is spread onto a drug in a high-shear mixer in order to achieve an intimate mixture of drug and hydrophilizing excipient; the resultant mixture is then dried and screened. Usually no benefit is accrued, however, when drug and hydrophilizing excipient are merely dry blended (see e.g. A. T. Florence et al., Modern Pharmaceutics, Volume 1, CRC Press 2010, page 543).

Various methods for the preparation of poloxamer-based drug delivery formulations are known which all have in common that the drug is intimately mixed with the poloxamer, such as the methods referred to as (i) direct solubilization of actives, (ii) thin film hydration, (iii) temperature-induced emulsification, (iv) solvent displacement, or (v) kinetically frozen micelles (A. M. Bodratti et al., J. Funct. Biomater. 2018, 9, 11, 1-24).

It is common general knowledge that surfactants such as poloxamers enhance the bioavailability of the drug, and that the granulation process can ensure an intimate mixture of drug and surfactant merely by the mixing energy of the process, or by putting both the drug and surfactant in the liquid used for granulating (see e.g. D. M. Parkih, Handbook of Pharmaceutical Granulation Technology, 4th ed., CRC Press 2021, page 136).

Surfactants are also used in solid drug dispersions as additives that alter dissolution via a mechanism other than the thermodynamic solubility advantage produced by an amorphous dispersion of a poorly soluble drug in a suitable matrix. A common approach is inclusion of a surfactant component such as poloxamer within the dispersion (i.e., intragranular) to enhance the wetting, disintegration, and dissolution of solid dispersed drug into solution (see e.g. A. Newman, Pharmaceutical Amorphous Solid Dispersions, Wiley 2015, page 345). Studies show that surfactants like poloxamer added extragranularly to solid dispersions typically have minimal effect on the drug release (J. P. Lakshman et al., Mol. Pharm. 5 (2008) 994-1002). However, when the surfactants are intimately mixed with solid dispersion, i.e. when surfactant, matrix polymer and drug are dissolved in common solvent and dried to yield solid dispersion, they have shown a positive effect on drug release. When surfactants are incorporated in the solid dispersion, the drug remains in the vicinity of the surfactant during dissolution creating microenvironment rich with surfactant increasing the drug solubility (S. P. Chaudhari et al., Journal of Drug Delivery Science and Technology 41 (2017) 68-77).

Therefore, formulating the surfactant e.g. poloxamer in the extragranular phase would typically not be considered as a straightforward approach for formulating Nilotinib.

Further, it has been surprisingly found that the surfactant in the extragranular phase may be homogeneously distributed by blending the granules with the extragranular phase, especially by geometric dilution. Without wishing to be bound to any scientific theory, it seems that surfactant may be blended with dry granulated granules containing the Nilotinib in a manner where surfactant deposits on the surface of the granules and develop their solubilizing activity when the granules come into contact with gastric juice. Thus, although the surfactant is not intimately mixed with the Nilotinib, the surfactant can nevertheless evolve its beneficial effect with regard to solubilizing the poorly soluble drug of Class IV and to ensure adequate in-vivo behavior. This can be achieved with both, non-micronized or micronized surfactant.

Still further, it has been surprisingly found that pharmaceutical compositions and dosage forms can be prepared that are stable and that contain in the intragranular phase anhydrous lactose in a mixture with Nilotinib, preferably anhydrous Nilotinib hydrochloride form T17, which according to WO 2017/064538 is said to be incompatible with anhydrous lactose. The present invention makes it possible to use anhydrous lactose and to keep it anhydrous throughout the shelf-life of the pharmaceutical composition.

Furthermore, it has been surprisingly found that pharmaceutical compositions can be prepared by an anhydrous granulation process (i.e. dry granulation) using surfactant such as poloxamer in the extragranular phase and nonetheless ensuring satisfactory and decent in vivo data with acceptable inter subject variability.

Moreover, it has been found that the employed form of Nilotinib, preferably anhydrous Nilotinib hydrochloride form T17 or Nilotinib hydrochloride monohydrate form B, can be maintained during an anhydrous granulation process (i.e. dry granulation).

A first aspect of the invention relates to a pharmaceutical composition comprising
(i) granules comprising Nilotinib, preferably in crystalline form, or a physiologically acceptable salt and/or solvate thereof, preferably crystalline anhydrous Nilotinib hydrochloride or crystalline Nilotinib hydrochloride monohydrate; and
(ii) an extragranular phase;
wherein the pharmaceutical composition comprises a surfactant which is contained in the extragranular phase, the granules, or both; preferably a poloxamer, more preferably non-micronized or micronized poloxamer.

Unless expressly stated otherwise, all percentages are weight percent (wt.-%).

Unless expressly stated otherwise, all editions and supplements of pharmacopoeia refer to the version that is valid on Jan. 1, 2021.

Unless expressly stated otherwise, “essentially consisting of” means at least 95 wt.-%, preferably at least 98 wt.-%, more preferably at least 99 wt.-%.

As used herein, the term “anhydrous” refers to a crystalline form having less than about 2 wt.-% total water (bound and unbound).

As used herein, the term “monohydrate” refers to a crystalline form having at least one molecule of water per molecule of Nilotinib (bound and unbound).

The pharmaceutical composition according to the invention comprises an intragranular phase and an extragranular phase, wherein the granules comprising Nilotinib form the intragranular phase.

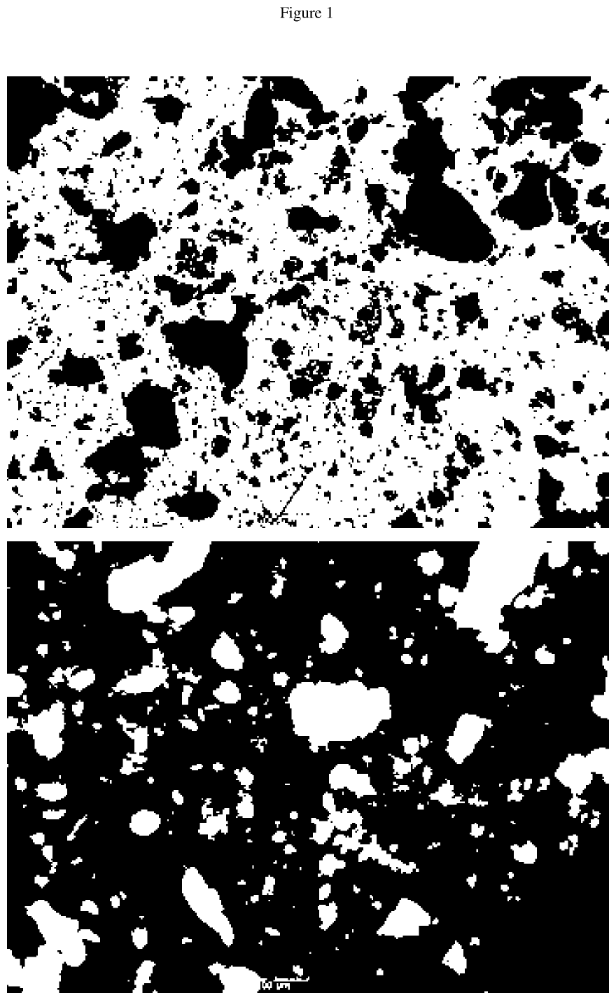

FIGS. 1 to 4 show photographs (10× magnitude) of various pharmaceutical formulations which were prepared by blending different types of poloxamer (coarse or micronized) with granules under different blending conditions (direct addition vs. geometric dilution). In each case the upper photograph was taken with polarized light, the lower photograph with non-polarized light.

FIG. 1 shows a comparative pharmaceutical formulation where the granules were blended with non-micronized poloxamer by direct addition.

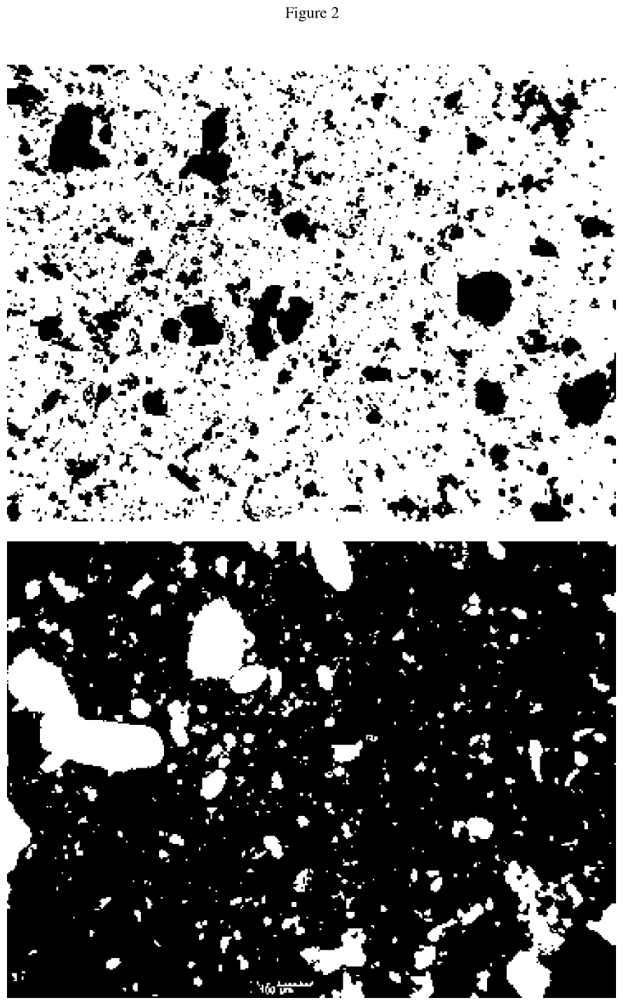

FIG. 2 shows another comparative pharmaceutical formulation where the granules were blended with micronized poloxamer by direct addition.

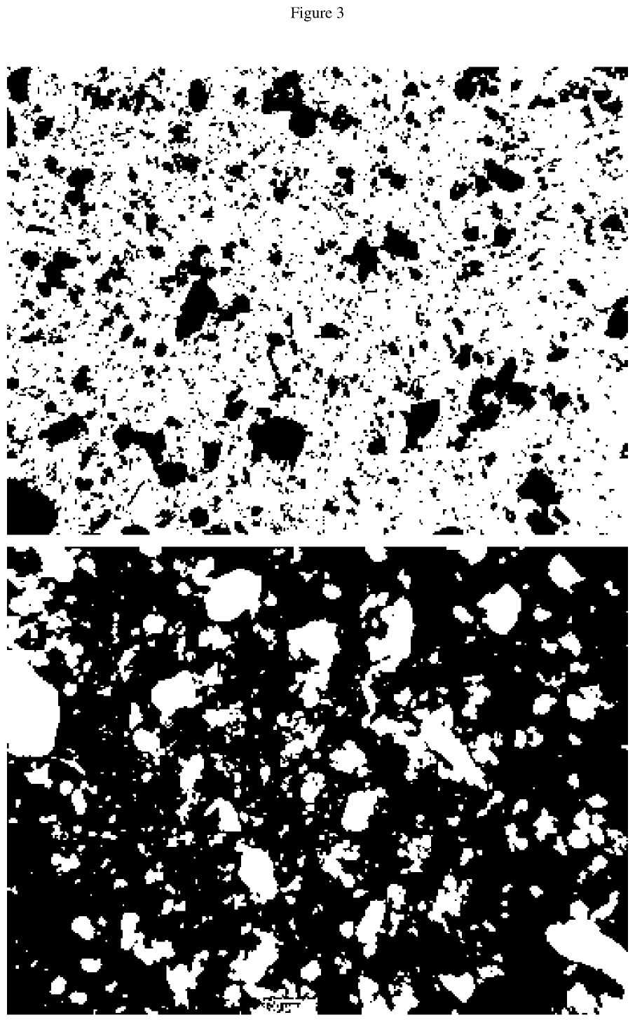

FIG. 3 shows an inventive pharmaceutical formulation where the granules were blended with non-micronized poloxamer by geometric dilution.

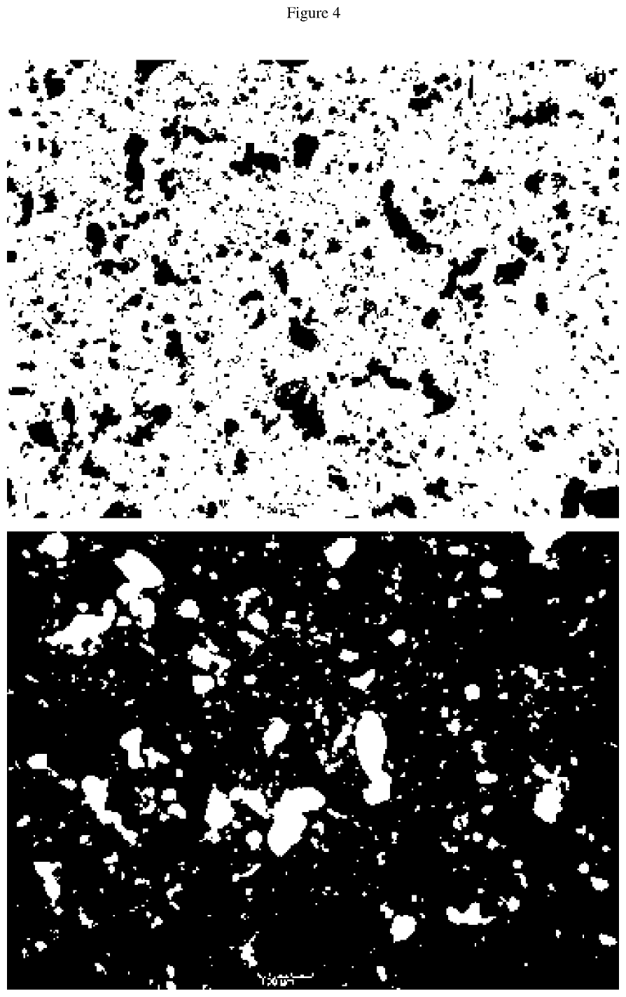

FIG. 4 shows another inventive pharmaceutical formulation where the granules were blended with micronized poloxamer by geometric dilution.

The pharmaceutical composition according to the invention comprises a surfactant that is preferably contained in the extragranular phase. Preferably, essentially the total amount of surfactant that is contained in the composition is contained in the extragranular phase.

In other preferred embodiments, the surfactant is contained in the granules, i.e. in the intragranular phase. Preferably, essentially the total amount of surfactant that is contained in the composition is contained in the granules.

In still other preferred embodiments, a first fraction of the surfactant is contained in the granules and a second fraction of the surfactant is contained in the extragranular phase. In these preferred embodiments, the relative weight ratio of the first fraction of the surfactant to the second fraction of the surfactant is preferably within the range of from 90:1 to 1:90, or from 80:1 to 1:80, or from 70:1 to 1:70, or from 60:1 to 1:60, or from 50:1 to 1:50, or from 40:1 to 1:40, or from 30:1 to 1:30, or from 20:1 to 1:20, or from 10:1 to 1:10.

Preferably, the relative weight ratio of the first fraction of the surfactant to the second fraction of the surfactant is within the range of from 90:1 to 70:1, or from 80:1 to 60:1, or from 70:1 to 50:1, or from 60:1 to 40:1, or from 50:1 to 30:1, or from 40:1 to 20:1, or from 30:1 to 10:1, or from 20:1 to 1:1.

Preferably, the relative weight ratio of the first fraction of the surfactant to the second fraction of the surfactant is within the range of from 1:70 to 1:90, or from 1:60 to 1:80, or from 1:50 to 1:70, or from 1:40 to 1:60, or from 1:30 to 1:50, or from 1:20 to 1:40, or from 1:10 to 1:30, or from 1:1 to 1:20.

In preferred embodiments, the surfactant is solid but not micronized, i.e. a coarse particulate material. According to the invention, the non-micronized surfactant is homogeneously distributed over the extragranular phase, the granules, or both of the pharmaceutical composition; preferably the extragranular phase. Preferably, in the course of manufacture of the pharmaceutical composition, the non-micronized surfactant is added by geometric dilution thereby achieving the desired degree of homogeneity of its distribution.

In other preferred embodiments, the surfactant is a micronized surfactant. Micronized surfactants are known to the skilled person and commercially available, e.g. micronized poloxamers. For the purpose of the specification, “micronized” refers to a powder having a weight average particle size on the micrometer scale. According to the invention, the micronized surfactant is also homogeneously distributed over the extragranular phase, the granules, or both of the pharmaceutical composition; preferably the extragranular phase. Preferably, in the course of manufacture of the pharmaceutical composition, the micronized surfactant is added by geometric dilution thereby achieving the desired degree of homogeneity of its distribution.

Preferably, the surfactant is homogeneously distributed such that the relative standard deviation (RSD) of the surfactant content in a multitude of samples of the pharmaceutical composition according to the invention, preferably in a multitude of 5 samples, more preferably in a multitude of 10 samples, is not more than 10.9%, preferably more than 10.6%, more preferably more than 10.3%, still more preferably more than 10.0%, yet more preferably more than 9.7%, even more preferably more than 9.4%, most preferably more than 9.1%, and in particular more than 8.8%. Preferably, when performing the test, one sample has a weight of about 1.0 g. Preferably, the surfactant is a poloxamer and the poloxamer content is quantified by HPLC, more preferably by HPLC-RI, in particular in accordance with Y. Mao et al., Quantitation of poloxamers in pharmaceutical formulations using size exclusion chromatography and colorimetric method, Journal of Pharmaceutical and Biomedical Analysis, 35 (2004) 1127-1142.

In preferred embodiments, the micronized surfactant has a weight average particle size as measured by sieve analysis in accordance with Ph. Eur. 2.9.38, of 50±40 μm, preferably 50±35 μm, more preferably 50±30 μm, still more preferably 50±25 μm, yet more preferably 50±20 μm, even more preferably 50±15 μm, most preferably 50±10 μm, and in particular 50±5 μm.

In preferred embodiments, the micronized surfactant has a particle size distribution as measured by sieve analysis in accordance with Ph. Eur. 2.9.38, wherein not more than 10 wt.-% retain on a sieve having a size of ≤106 μm, and not more than 50 wt.-% retain on a sieve having a size of ≤53 μm.

While it is contemplated that the surfactant may be anionic, cationic or amphoteric, preferably the surfactant is a nonionic surfactant, preferably an ethoxylated surfactant or a polyol-based surfactant; preferably an ether or an ester.

In preferred embodiments, the surfactant is selected from the group consisting of (a) poloxamers; (b) ethoxylated fatty alcohols; (c) alkyl phenol ethoxylates; (d) polyoxyethylene fatty acid esters; (e) polyoxyethylene esters of alpha-tocopheryl succinate; (f) polyoxyethylene glycerol fatty acid esters; (g) reaction products of a natural or hydrogenated castor oil and ethylene oxide; (h) partial fatty acid esters of sorbitan; (i) partial fatty acid esters of polyoxyethylene sorbitan; preferably fatty acid monoester of polyoxyethylene sorbitan, fatty acid diester of polyoxyethylene sorbitan, or a fatty acid triester of polyoxyethylene sorbitan; (j) fatty acid esters or glycerol; preferably glycerol fatty acid monoester, glycerol fatty acid diester, or glycerol fatty acid triester; (k) polyglycolyzed glycerides; (1) partial fatty acid esters of pentaerythritol; (m) partial fatty acid esters of saccharose; and (n) partial fatty acid esters of polyglycerol.

Preferably, the surfactant is a solid at room temperature. For the purpose of the specification, surfactants that are solid at room temperature (i.e. 23° C.) include surfactants that are waxy materials.

Preferably, the surfactant has a HLB value within the range of 29±8, preferably 29±7, more preferably 29±6, still more preferably 29±5, yet more preferably 29±4, even more preferably 29±3, most preferably 29±2, and in particular 29±1.

In particularly preferred embodiments, the surfactant is a poloxamer; preferably poloxamer 188 or poloxamer 407. Poloxamer 188 and 407 are commercially available, e.g. under the trade name Kolliphor®, Lutrol® F68, Pluronic® F-68, and Synperonic® F68, and the like.

Poloxamer 188 is known to the skilled person and commercially available. Poloxamer 188 satisfies the following formula HO—[—CH₂CH₂—O-]ₐ-[—CH₂CH(CH₃)—O-]_{b}-[—CH₂CH₂—O-]ₐ—H, wherein index a is approximately 79 and index b is approximately 28, whereas the proportion of polyoxyethylene is approximately 70 wt.-%. In poloxamer 407, index a is approximately 101 and index b is approximately 56, whereas the proportion of polyoxyethylene is approximately 80 wt.-%.

Preferably, essentially the total amount of surfactant that is contained in the composition according to the invention is contained in the extragranular phase, i.e. the granules preferably contain no surfactant.

Preferably, the content of the surfactant is within the range of from 0.5 to 1.5 wt.-%, preferably 0.70±0.20 wt.-%, more preferably 0.80±0.20 wt.-%, still more preferably 0.90±0.20 wt.-%, yet more preferably 1.00±0.20 wt.-%, relative to the total weight of the composition.

The pharmaceutical composition according to the invention comprises Nilotinib (C₂₈H₂₂F₃N₇O, ATC L01EA03), i.e. 4-methyl-N-[3-(4-methyl-1H-imidazol-1-yl)-5-(trifluoromethyl)phenyl]-3-{[4-(pyridin-3-yl)pyrimidin-2-yl]amino} benzamide:

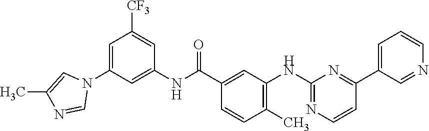

Unless expressly stated otherwise, all percentages and dosages are expressed as equivalents based upon the weight of Nilotinib in its non-salt and non-solvate form, i.e. C₂₈H₂₂F₃N₇O.

Preferably, the Nilotinib is crystalline.

Preferably, the pharmaceutical composition according to the invention comprises no amorphous Nilotinib, physiologically acceptable salt and/or solvate thereof.

Preferably, the Nilotinib is anhydrous or a hydrate.

Preferably, the Nilotinib is anhydrous; preferably a Nilotinib ansolvate.

Preferably, the Nilotinib is a hydrate; preferably a monohydrate.

Preferably, the Nilotinib is a Nilotinib hydrochloride salt.

In particularly preferred embodiments of the invention, the Nilotinib is crystalline anhydrous Nilotinib hydrochloride salt; preferably Nilotinib HCl form T17. Nilotinib HCl form T17 is described e.g. in WO 2010/054056.

Nilotinib HCl form T17 is characterized by data selected from the group consisting of: (i) an x-ray powder diffraction pattern having peaks at 5.7, 9.8, 15.0, 15.8 and 17.3 degrees two theta±0.2 degrees two theta; preferably having additional peaks at 7.5, 11.4, 18.6, 19.6 and 20.7 degrees two theta±0.2 degrees two theta, or at 7.6, 11.4, 18.7, 19.7 and 20.7 degrees two theta±0.2 degrees two theta; (ii) a solid-state 13C NMR spectrum with signals at 113.1, 133.1, 160.9±0.2 ppm; (iii) a solid-state 13C NMR spectrum having chemical shifts differences between the signal exhibiting the lowest chemical shift and another in the chemical shift range of 100 to 180 ppm of 9.2, 29.2 and 57.0 60.1 ppm, wherein the signal exhibiting the lowest chemical shift in the chemical shift area of 100 to 180 ppm is typically at about 103.9±0.2 ppm; and combinations thereof.

Anhydrous Nilotinib hydrochloride is characterized as a Class IV compound (low/moderate aqueous solubility and low permeability) according to the Biopharmaceutical Classification System (BCS).

In other preferred embodiments of the invention, the Nilotinib is crystalline Nilotinib hydrochloride monohydrate salt; preferably Nilotinib HCl form B. Nilotinib HCl form B is described e.g. in WO 2007/015870.

Nilotinib HCl form B is characterized by an x-ray powder diffraction pattern having at least one maxima selected from 7.2, 9.2, 11.4, 12.0, 12.3, 14.6, 14.8, 15.7, 17.6, 19.2, 19.5, 20.5, 22.0, 23.4, 23.9, 25.0, 25.5, 25.9 and 27.0 degrees two theta.

Preferably, the content of Nilotinib is within the range of 50±35 wt.-%, preferably 50±30 wt.-%, more preferably 50±25 wt.-%, still more preferably 50±20 wt.-%, yet more preferably 50±15 wt.-%, even more preferably 50±10 wt.-%, most preferably 50±5.0 wt.-%, and in particular 50±2.5 wt.-%, relative to the total weight of the composition and expressed as equivalent weight of Nilotinib in its non-salt non-solvate form.

The granules and/or the extragranular phase of the pharmaceutical composition according to the invention may comprise one or more pharmaceutical excipients. Preferably, the pharmaceutical excipients are selected from diluents (fillers), disintegrants, glidants, lubricants, surfactants, binders, antioxidants, preservatives, colorants, pigments, dyes, pH modifiers, and the like.

Preferably, the granules of the pharmaceutical composition according to the invention are composed of a granulated mixture of all ingredients, i.e. of Nilotinib and all intragranular excipients, in neat form, but is not composed of a mixture of a preformulated blend with intragranular excipients. For example, the granules of the pharmaceutical composition according to the invention are preferably not composed of a spray dried formulation comprising Nilotinib and one or more excipients (preformulated blend) in admixture with further excipients.

Preferably, the granules of the composition according to the invention comprise a diluent.

Preferably, the diluent is selected from the group consisting of lactose, glucose, sucrose, sorbitol, mannitol, dextrates, dextrin, dextrose, microcrystalline cellulose, and powdered cellulose; preferably lactose.

In particularly preferred embodiments, the diluent is anhydrous; preferably anhydrous lactose.

Preferably, the content of the diluent is within the range of 40±35 wt.-%, preferably 40±30 wt.-%, more preferably 40±25 wt.-%, still more preferably 40±20 wt.-%, yet more preferably 40±15 wt.-%, even more preferably 40±10 wt.-%, most preferably 40±5.0 wt.-%, and in particular 40±2.5 wt.-%, relative to the total weight of the composition.

Preferably, essentially the total amount of diluent that is contained in the composition according to the invention is contained in the granules, i.e. the extragranular phase preferably contains no diluent. Preferably, the granules of the composition according to the invention comprise a disintegrant.

Preferably, the disintegrant is selected from the group consisting of cross-linked polyvinyl pyrrolidone, cross-linked sodium carboxymethylcellulose, cross-linked calcium carboxymethylcellulose, pregelatinized starch, clay, cellulose, soy polysaccharides, alginates, and gums.

In particularly preferred embodiments, the disintegrant is cross-linked polyvinyl pyrrolidone; preferably crospovidone according to Ph. Eur. crospovidone monograph type A. Examples and commercial grades of crospovidone can be used, e.g. Kollidon® CL, Kollidon® CL-M, Polyplasdone® XL, Polyplasdone® XL-10, Polyplasdone® Ultra. Preferably crospovidone used in the composition has a mean particle size of less than 150 μm.

Preferably, the disintegrant has a weight average particle size as measured by sieve analysis in accordance with Ph. Eur. 2.9.38, of 125±50 μm, preferably 125±45 μm, more preferably 125±40 μm, still more preferably 125±35 μm, yet more preferably 125±30 μm, even more preferably 125±25 μm, most preferably 125±20 μm, and in particular 125±15 μm.

Preferably, the content of the disintegrant is within the range of 4.0±3.5 wt.-%, preferably 4.0±3.0 wt.-%, more preferably 4.0±2.5 wt.-%, still more preferably 4.0±2.0 wt.-%, yet more preferably 4.0±1.6 wt.-%, even more preferably 4.0±1.4 wt.-%, most preferably 4.0±1.2 wt.-%, and in particular 4.0±1.0 wt.-%, relative to the total weight of the composition.

Preferably, essentially the total amount of disintegrant that is contained in the composition according to the invention is contained in the granules, i.e. the extragranular phase preferably contains no disintegrant.

Preferably, the pharmaceutical composition according to the invention comprises a glidant.

In preferred embodiments, the granules comprise a glidant. In other preferred embodiments, the extragranular phase comprises a glidant. In further preferred embodiments, the granules as well as the extragranular phase comprise a glidant, which is preferably the same glidant.

Preferably, the glidant is selected from the group consisting of colloidal silica, magnesium trisilicate, starch, talc, tribasic calcium phosphate, magnesium carbonate, magnesium oxide and powdered cellulose; preferably colloidal silica.

In particularly preferred embodiments, the glidant is anhydrous colloidal silica.

Preferably, the content of the glidant is within the range of 1.0±0.9 wt.-%, preferably 1.0±0.8 wt.-%, more preferably 1.0±0.7 wt.-%, still more preferably 1.0±0.6 wt.-%, yet more preferably 1.0±0.5 wt.-%, even more preferably 1.0±0.4 wt.-%, most preferably 1.0±0.3 wt.-%, and in particular 1.0±0.2 wt.-%, relative to the total weight of the composition.

In preferred embodiments, a first fraction of the glidant is contained in the granules and a second fraction (the remainder) of the glidant is contained in the extragranular phase.

Preferably, the pharmaceutical composition according to the invention comprises a lubricant.

In preferred embodiments, the granules comprise a lubricant. In other preferred embodiments, the extragranular phase comprises a lubricant. In further preferred embodiments, the granules as well as the extragranular phase comprise a lubricant, which is preferably the same lubricant.

Preferably, the lubricant is selected from the group consisting of magnesium stearate, aluminum stearate, calcium stearate, polyethylene glycol, glyceryl behenate, stearic acid, hydrogenated castor oil, glyceryl monostearate and sodium stearyl fumarate.

In particularly preferred embodiments, the lubricant is magnesium stearate.

Preferably, the content of the lubricant is within the range of 1.5±1.3 wt.-%, preferably 1.5±1.1 wt.-%, more preferably 1.5±0.9 wt.-%, still more preferably 1.5±0.7 wt.-%, yet more preferably 1.5±0.5 wt.-%, even more preferably 1.5±0.3 wt.-%, most preferably 1.5±0.2 wt.-%, and in particular 1.5±0.1 wt.-%, relative to the total weight of the composition.

In preferred embodiments, a first fraction of the lubricant is contained in the granules and a second fraction (the remainder) of the lubricant is contained in the extragranular phase.

In preferred embodiments, the pharmaceutical composition according to the invention comprises no povidone; preferably no binder selected from the group consisting of povidone, microcrystalline cellulose, hydroxyethylcellulose, hydroxypropylcellulose, and hydroxypropylmethylcellulose.

In preferred embodiments, the granules essentially consist of Nilotinib, preferably in crystalline form, or a physiologically acceptable salt and/or solvate thereof, preferably crystalline anhydrous Nilotinib hydrochloride or crystalline Nilotinib hydrochloride monohydrate, diluent, disintegrant, optionally glidant, and optionally lubricant.

In other preferred embodiments, the granules essentially consist of Nilotinib, preferably in crystalline form, or a physiologically acceptable salt and/or solvate thereof, preferably crystalline anhydrous Nilotinib hydrochloride or crystalline Nilotinib hydrochloride monohydrate, diluent, disintegrant, optionally glidant, optionally lubricant, and optionally surfactant.

In preferred embodiments, the extragranular phase contains no lactose; preferably no diluent selected from the group consisting of lactose, glucose, sucrose, sorbitol, mannitol, dextrates, dextrin, dextrose, microcrystalline cellulose, and powdered cellulose.

In other preferred embodiments, the extragranular phase essentially consists of glidant and lubricant.

In preferred embodiments, the extragranular phase essentially consists of surfactant, glidant and lubricant.

In preferred embodiments, the granules are dry granulated.

In particularly preferred embodiments of the pharmaceutical composition according to the invention
(i) the granules comprise or essentially consist of
   crystalline anhydrous Nilotinib hydrochloride or crystalline Nilotinib hydrochloride monohydrate;
   diluent, preferably anhydrous lactose;
   disintegrant, preferably cross-linked polyvinylpyrrolidone;
   a fraction of glidant; preferably anhydrous colloidal silica; and
   a fraction of lubricant; preferably magnesium stearate; and
(ii) the extragranular phase comprises or essentially consists of
   poloxamer; preferably poloxamer 188 and/or micronized poloxamer;
   the remainder of glidant; preferably anhydrous colloidal silica;
   the remainder of lubricant; preferably magnesium stearate.

In other preferred embodiments of the pharmaceutical composition according to the invention
(i) the granules comprise or essentially consist of
   crystalline anhydrous Nilotinib hydrochloride or crystalline Nilotinib hydrochloride monohydrate;
   diluent, preferably anhydrous lactose;
   disintegrant, preferably cross-linked polyvinylpyrrolidone;
   a fraction of glidant; preferably anhydrous colloidal silica;
   a fraction of lubricant; preferably magnesium stearate; and
   poloxamer; preferably poloxamer 188 and/or micronized poloxamer; and
(ii) the extragranular phase comprises or essentially consists of
   the remainder of glidant; preferably anhydrous colloidal silica;
   the remainder of lubricant; preferably magnesium stearate.

In still other preferred embodiments of the pharmaceutical composition according to the invention
(i) the granules comprise or essentially consist of
   crystalline anhydrous Nilotinib hydrochloride or crystalline Nilotinib hydrochloride monohydrate;
   diluent, preferably anhydrous lactose;
   disintegrant, preferably cross-linked polyvinylpyrrolidone;
   a fraction of glidant; preferably anhydrous colloidal silica; and
   a fraction of lubricant; preferably magnesium stearate;
   a fraction of poloxamer; preferably poloxamer 188 and/or micronized poloxamer; and
(ii) the extragranular phase comprises or essentially consists of
   the remainder of poloxamer; preferably poloxamer 188 and/or micronized poloxamer;
   the remainder of glidant; preferably anhydrous colloidal silica;
   the remainder of lubricant; preferably magnesium stearate.

Preferably, the pharmaceutical composition according to the invention does not contain citric acid or a salt thereof.

Preferably, the pharmaceutical composition according to the invention does not contain an organic acid selected from acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, glutamic acid, aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid and ascorbic acid or a salt thereof; preferably no organic acid at all or any salt thereof.

Preferably, the granules that are contained in the pharmaceutical composition according to the invention have a size such that they pass a sieve having a mesh size of 1.0 to 2.0 mm but do not pass a sieve having a mesh size of 0.5 mm; preferably 0.6 mm, more preferably 0.7 mm, still more preferably 0.8 mm.

Preferably, the granules have an essentially spherical shape, although other shapes are contemplated as well.

Another aspect of the invention relates to a process for the preparation of a pharmaceutical composition according to the invention as described above comprising the step of dry granulating a formulation comprising the Nilotinib, preferably in crystalline form, or a physiologically acceptable salt and/or solvate thereof, preferably crystalline anhydrous Nilotinib hydrochloride or crystalline Nilotinib hydrochloride monohydrate.

Dry granulating according to the invention involves either slugging or roller compaction.

Slugging is a compaction process in which the material to be processed is compressed to a large compressed mass, or “slug,” which is further milled to form granules and are finally filled into capsules or compressed into tablets. The slugging process typically includes (i) sifting and blending of dry mix ingredients; (ii) compressing the blend of step (i) to obtain slugs; (iii) milling and sifting the step of (ii) to obtain granules; (iv) lubricating and blending the granules of step (iii), and finally filling into capsules or compressing into tablets.

Roller compaction is a process by which two or more solid materials are compacted between rotating rolls, desirably counter-rotating rolls, to form solid ribbons/compacts. The resulted compacts are typically sized into granules by milling to modify the desired particle size; and optionally the granules are mixed with one or more of pharmaceutically acceptable excipients to form the composition.

The process for process according to the invention preferably comprises compacting Nilotinib, preferably in crystalline form, or a physiologically acceptable salt and/or solvate thereof, preferably crystalline anhydrous Nilotinib hydrochloride or crystalline Nilotinib hydrochloride monohydrate, alone or mixing with one or more of pharmaceutically acceptable excipient(s) by roller compactor or slugging; sizing the compacts or slugs into granules by milling; and mixing the granules with one or more of pharmaceutically acceptable excipients to form the final composition. The mixing process preferably includes: (i) sifting and blending of Nilotinib, preferably in crystalline form, or a physiologically acceptable salt and/or solvate thereof, preferably crystalline anhydrous Nilotinib hydrochloride or crystalline Nilotinib hydrochloride monohydrate, with one or more pharmaceutically acceptable excipients to form a dry mixture (pharmaceutical composition); (ii) followed by filling the dry mixture of step (i) into capsules (pharmaceutical dosage form).

Preferably, the process comprises the steps of
(a) providing a formulation comprising Nilotinib, preferably in crystalline form, or a physiologically acceptable salt and/or solvate thereof, preferably crystalline anhydrous Nilotinib hydrochloride or crystalline Nilotinib hydrochloride monohydrate, optionally diluent, optionally disintegrant, optionally a fraction of glidant, optionally a fraction of lubricant, and optionally surfactant or a fraction of surfactant;
(b) dry granulating the formulation thereby obtaining granules;
(c) optionally, sieving the granules;
(d) optionally, providing a surfactant formulation comprising or essentially consisting of surfactant or the remainder of surfactant, preferably poloxamer; and
(e) optionally, blending the surfactant formulation and the granules.

In preferred embodiments, the process comprises the steps of
(a) providing a formulation comprising Nilotinib, preferably in crystalline form, or a physiologically acceptable salt and/or solvate thereof, preferably crystalline anhydrous Nilotinib hydrochloride or crystalline Nilotinib hydrochloride monohydrate, optionally diluent, optionally disintegrant, optionally a fraction of glidant, and optionally a fraction of lubricant;
(b) dry granulating the formulation thereby obtaining granules;
(c) optionally, sieving the granules;
(d) providing a surfactant formulation comprising or essentially consisting of surfactant, preferably poloxamer; and
(e) blending the surfactant formulation and the granules.

In other preferred embodiments, the process comprises the steps of
(a) providing a formulation comprising Nilotinib, preferably in crystalline form, or a physiologically acceptable salt and/or solvate thereof, preferably crystalline anhydrous Nilotinib hydrochloride or crystalline Nilotinib hydrochloride monohydrate, optionally diluent, optionally disintegrant, optionally a fraction of glidant, optionally a fraction of lubricant, and surfactant;
(b) dry granulating the formulation thereby obtaining granules;
(c) optionally, sieving the granules.

In still other preferred embodiments, the process comprises the steps of
(a) providing a formulation comprising Nilotinib, preferably in crystalline form, or a physiologically acceptable salt and/or solvate thereof, preferably crystalline anhydrous Nilotinib hydrochloride or crystalline Nilotinib hydrochloride monohydrate, optionally diluent, optionally disintegrant, optionally a fraction of glidant, optionally a fraction of lubricant, and a fraction of surfactant;
(b) dry granulating the formulation thereby obtaining granules;
(c) optionally, sieving the granules;
(d) providing a surfactant formulation comprising or essentially consisting of the remainder of surfactant, preferably poloxamer; and
(e) blending the surfactant formulation and the granules.

In step (a) of the process according to the invention, a formulation is provided that comprises Nilotinib, preferably in crystalline form, or a physiologically acceptable salt and/or solvate thereof, preferably crystalline anhydrous Nilotinib hydrochloride or crystalline Nilotinib hydrochloride monohydrate, and optionally one or more of the following, preferably all of the following: diluent, disintegrant, a fraction of glidant, a fraction of lubricant and optionally surfactant or a fraction of surfactant.

All ingredients of the formulation are preferably weighed. Preferably, in a first sub-step, all excipients are mixed and sifted, and subsequently the Nilotinib, preferably in crystalline form, or a physiologically acceptable salt and/or solvate thereof, preferably crystalline anhydrous Nilotinib hydrochloride or crystalline Nilotinib hydrochloride monohydrate is added. The mixture is then preferably blended, e.g. at a rotational speed of the blender within the range of from 3 to 9 rpm, more preferably 5 to 7 rpm. The blending time is not particularly limited. Blending typically lasts 5 to 15 minutes, preferably 8 to 12 minutes.

In step (b) of the process according to the invention, the formulation provided in step (a) is dry granulated thereby obtaining granules.

Preferably, dry granulating is performed by roller compaction.

Preferably, roller compaction is performed at an average roller gap within the range of from 0.5 to 2.0 mm; preferably 1.0±0.2 mm.

Preferably, roller compaction is performed at a pressure within the range of from 20 to 40 bar; preferably 25 to 35 bar.

In optional step (c) of the process according to the invention, the granules are sieved thereby obtaining calibrated granules.

Preferably, sieving the granules in step (c) calibrates large granules not passing a sieve of size 1.60 mm as well as small granules passing a sieve of size 0.80 mm.

Optional steps (d) and (e) are preferably performed when no surfactant or a fraction of surfactant is provided in step (a). Optional steps (d) and (e) are preferably not performed when essentially the total amount of surfactant is provided in step (a). Optional step (e) is preferably performed after optional step (d), i.e. when optional step (d) is not performed, optional step (e) is preferably not performed either.

In optional step (d) of the process according to the invention, a surfactant formulation is provided that comprises or essentially consisting of surfactant or a fraction of surfactant, preferably poloxamer. Preferably, the surfactant formulation essentially consists of surfactant or a fraction of surfactant, preferably poloxamer.

In optional step (e) of the process according to the invention, the granules obtained in step (b), or preferably the calibrated granules obtained in optional step (c), are blended with the surfactant formulation provided in optional step (d).

Preferably, in optional step (e) in an initial sub-step the surfactant formulation is blended with a fraction of the granules, and wherein in subsequent sub-steps additional fractions of the granules are added to the blend (geometric dilution process).

Thus, the granules obtained in step (b), or preferably the calibrated granules obtained in optional step (c), are preferably divided into fractions, preferably having approximately the same weight. Preferably, the granules are divided into 2, 3, 4, 5, 6, 7, 8, 9, or 10 fractions. Preferably, in an initial sub-step the surfactant formulation is blended with a first fraction of the granules, in a subsequent sub-step a second fraction of the granules is added to the blend and the thus obtained mixture is further blended, in another subsequent sub-step a third fraction of the granules is added to the blend and the thus obtained mixture is further blended, and so on (geometric dilution process).

Preferably, optional step (e) comprises the sub-steps
(e₁) blending the surfactant formulation with a first fraction of the granules thereby obtaining a first blend; and optionally sieving the first blend;
(e₂) blending the first blend with a second fraction of the granules thereby obtaining a second blend; optionally sieving the second blend;
(e₃) optionally, blending the second blend with a third fraction of the granules thereby obtaining a third blend; optionally sieving the third blend;
(e₄) optionally, blending the third blend with a fourth fraction of granules thereby obtaining a fourth blend; optionally sieving the fourth blend; and
(e₅) optionally, blending the fourth blend with a fifth fraction of granules thereby obtaining a fifth blend; optionally sieving the fifth blend.

Preferably, every blending step, i.e. (e₁), (e₂), and so on is performed for 0.5 to 2 minutes, preferably 1 minute.

Preferably, in optional step (e) the surfactant is blended with the granules until a homogeneous distribution is achieved such that the relative standard deviation (RSD) of the surfactant content in a multitude of samples, preferably in a multitude of 5 samples, more preferably in a multitude of 10 samples, is not more than 10.9%, preferably more than 10.6%, more preferably more than 10.3%, still more preferably more than 10.0%, yet more preferably more than 9.7%, even more preferably more than 9.4%, most preferably more than 9.1%, and in particular more than 8.8%. Preferably, when performing the test, one sample has a weight of about 1.0 g. Preferably, the surfactant is a poloxamer and the poloxamer content is quantified by HPLC, more preferably by HPLC-RI.

Sieving the blends is preferably performed by means of a sieve having a size of 0.5 to 1.0 mm, e.g. 0.7 mm.

In preferred embodiments, the process comprises the additional steps of blending the granules obtained in step (b), the sieved granules obtained in step (c) or the product obtained in step (e)
(f) with the remainder of glidant; and optionally, sieving the thus obtained blend; and/or
(g) with the remainder of lubricant; and optionally, sieving the thus obtained blend.

In step (f) of the process according to the invention, the granules obtained in step (b), the sieved granules obtained in optional step (c) or the product obtained in optional step (e) is blended with glidant. Preferably, the glidant is the same glidant that is also contained in the granules, i.e. preferably the granules obtained in step (b), the sieved granules obtained in optional step (c), or the product obtained in optional step (e) is blended with the remainder of glidant.

The mixture is then preferably blended, e.g. at a rotational speed of the blender within the range of from 3 to 9 rpm, more preferably 5 to 7 rpm. The blending time is not particularly limited. Blending typically lasts 1 to 10 minutes, preferably 3 to 7 minutes.

In step (g) of the process according to the invention, the granules obtained in step (b), the sieved granules obtained in optional step (c), the product obtained in optional step (e) or the product obtained in step (f) is blended with lubricant. Preferably, the lubricant is the same lubricant that is also contained in the granules, i.e. preferably the granules obtained in step (b), the sieved granules obtained in optional step (c), the product obtained in optional step (e) or the product obtained in step (f) is blended with the remainder of lubricant.

The mixture is then preferably blended, e.g. at a rotational speed of the blender within the range of from 3 to 9 rpm, more preferably 5 to 7 rpm. The blending time is not particularly limited. Blending typically lasts 1 to 5 minutes, preferably 2 to 4 minutes.

Preferably, in the process according to the invention, the form of Nilotinib provided in step (a) is maintained in the product.

Another aspect of the invention relates to a pharmaceutical composition that is obtainable by the process according to the invention as described above.

Another aspect of the invention relates to a pharmaceutical dosage form comprising the pharmaceutical composition according to the invention as described above.

Preferably, the pharmaceutical dosage form according to the invention is devoted for oral administration.

Preferably, the pharmaceutical dosage form according to the invention is a capsule; preferably a hard gelatin capsule. Preferably, the pharmaceutical composition according to the invention is contained in the capsule in form of a loose capsule filling, i.e. not in compacted form.

The dose of Nilotinib that is contained in the pharmaceutical dosage form is not particularly limited. In preferred embodiments, the pharmaceutical dosage form contains a dose of 50 mg, 100 mg, 150 mg, 200 mg, or 250 mg, in each case expressed as equivalent weight based upon the non-salt non-solvate form of Nilotinib.

The recommended dose is (i) 300 mg twice daily in newly diagnosed patients with chronic myelogenous leukemia (CML) in the chronic phase, or (ii) 400 mg twice daily in patients with chronic or accelerated phase chronic myelogenous leukemia (CML) with resistance or intolerance to prior therapy.

The pharmaceutical dosage form according to the invention preferably provides immediate release of Nilotinib. Preferably, when determining in vitro dissolution in accordance with Ph. Eur. and USP, basket method, 100 rpm, 1000 mL 0.1N HCl, 37° C., after 30 minutes at least 70 wt.-%, preferably at least 80 wt.-% of the Nilotinib that was originally contained in the dosage form have been released.

Another aspect of the invention relates to the pharmaceutical dosage form according to the invention as described above for use in the treatment of cancer. The invention accordingly also relates to the use of Nilotinib of a physiologically acceptable salt and or solvate thereof for the manufacture of a pharmaceutical dosage form according to the invention as described above for treating cancer. The invention accordingly also relates to a method for treating cancer comprising administering to a subject in need thereof the pharmaceutical dosage form according to the invention as described above.

Preferably, the cancer is chronic myeloid leukemia or a gastrointestinal stromal tumor.

Preferably, the patients to be treated are adult and pediatric patients with newly diagnosed Philadelphia chromosome positive chronic myelogenous leukemia (CML) in the chronic phase; adult patients with chronic phase and accelerated phase Philadelphia chromosome positive CML with resistance or intolerance to prior therapy including Imatinib; or pediatric patients with chronic phase Philadelphia chromosome positive CML with resistance or intolerance to prior therapy including Imatinib.

Preferably, administration proceeds orally; preferably once daily or twice daily; more preferably twice daily.

Peak concentrations of Nilotinib are preferably reached 3 hours after oral administration of the pharmaceutical dosage form according to the invention. In preferred embodiments, the dosage form for use according to the invention upon oral administration provides an Coefficient of Variation (inter-patient variability) of cₘₐₓ of not more than 50%, preferably not more than 45%; and/or of AUC_{0-∞} of not more than 55%, preferably not more than 50%. Methods for determining cₘₐₓ, AUC and the Coefficient of Variation thereof are known to the skilled person and according to the invention are determined in accordance with guidance of regulatory authorities (see e.g. Guidance for Industry, Q2B Validation of Analytical Procedures: Methodology). Preferably, these parameters are determined in accordance with X. Tian et al., Clinical Pharmacokinetic and Pharmacodynamic Overview of Nilotinib, a Selective Tyrosine Kinase Inhibitor, The Journal of Clinical Pharmacology, 2018, 58(12) 1533-1540.

The following examples further illustrate the invention but are not to be construed as limiting its scope.

### Examples 1-3 (Inventive)

Three pharmaceutical dosage forms having a dose strength of Nilotinib of 50 mg, 150 mg and 200 mg were prepared from different amounts of the same pharmaceutical composition. The amounts are compiled in the table here below:

| | | Quantity per capsule | | | |
|---|---|---|---|---|---|
| | | | 1 | 2 | 3 |
| Composition | Function | [%] | [mg] | [mg] | [mg] |
| Nilotinib hydrochloride | Drug | 53.45 | 53.45 (50) | 160.35 (150) | 213.80 (200) |
| (corresponding to Nilotinib base) | | | | | |
| Lactose, anhydrous | Diluent | 39.37 | 39.37 | 118.12 | 157.49 |
| Crospovidone (Type A) | Disintegrant | 3.98 | 3.98 | 11.93 | 15.91 |
| Silica, colloidal anhydrous | Glidant | 1.00 | 1.00 | 3.00 | 4.00 |
| Magnesium stearate | Lubricant | 1.50 | 1.50 | 4.50 | 6.00 |
| Poloxamer P188 | Surfactant | 0.70 | 0.70 | 2.10 | 2.80 |
| total | | 100.00 | 100.00 | 300.00 | 400.00 |

Anhydrous colloidal silica (glidant) and magnesium stearate (lubricant) were each divided into a first fraction and into a second fraction (remainder), 50:50 w/w.

The pharmaceutical composition consisted of an intragranular phase prepared by dry granulation (roller compaction) and an extragranular phase, as compiled in the following table:

| | | |
|---|---|---|
| intragranular | Nilotinib hydrochloride | Drug |
| | (corresponding to Nilotinib base) | |
| | Lactose, anhydrous | Diluent |
| | Crospovidone (Type A) | Disintegrant |
| | Silica, colloidal anhydrous Part 1 | Glidant |
| | Magnesium stearate Part 1 | Lubricant |
| extragranular | Poloxamer P188 | Surfactant |
| | Silica, colloidal anhydrous Part 2 | Glidant |
| | Magnesium stearate Part 2 | Lubricant |

Anhydrous lactose, crospovidone, the first fraction of anhydrous colloidal silica and the first fraction of magnesium stearate were weighed and sifted over a sieve having a net size of 0.7 mm. Crystalline anhydrous Nilotinib hydrochloride (form T17) was added and the mixture was blended in a rotational blender (Böhle PM 400) for 10 minutes at 6 rpm.

The blend was roller compacted (Alexanderwerk WP120N) at a hydraulic pressure of about 30 bar and an average roller gap of about 1 mm. The dry granules were sieved, first net sieve 1.60 mm, second net sieve 0.80 mm.

The thus obtained calibrated granules were divided into five fractions (geometric dilution process). Poloxamer 188 was mixed with a first fraction of the calibrated granules, sieved through a 0.7 mm sieve and blended for 1 minute. A second fraction of the calibrated granules was added, sieved through a 0.7 mm sieve and blended for 1 minute. A third fraction of the calibrated granules was added, sieved through a 0.7 mm sieve and blended for 1 minute. A fourth fraction of the calibrated granules was added, sieved through a 0.7 mm sieve and blended for 1 minute. A fifth and final fraction of the calibrated granules was added, sieved through a 0.7 mm sieve and blended for 1 minute.

The thus obtained product was mixed with the second fraction (remainder) of anhydrous colloidal silica (glidant) and the mixture was blended in a rotational blender (Böhle PM 400) for 5 minutes at 6 rpm.

The thus obtained pre-blend was mixed with the second fraction (remainder) of magnesium stearate (lubricant) and the mixture was blended in a rotational blender (Böhle PM 400) for 3 minutes at 6 rpm.

The thus obtained pharmaceutical composition was filled into hard gelatin capsules of different size at the desired dose strength.

### Examples 4 and 5 (Comparative) and Examples 6 and 7 (Inventive)

Four pharmaceutical compositions were prepared under the same conditions from the same starting materials in the same individual amount (content of poloxamer 0.7 wt.-%, see Example 1).

The only difference was that two different commercial poloxamers having different particle size (non-micronized [Kolliphor® P 188] vs. micronized [Kolliphor® P 188 micro]) were used and that the poloxamer was blended with the granules (that had been prepared by dry granulation under identical conditions) under different blending conditions (direct addition vs. geometric dilution):

| | comparative | | inventive | |
|---|---|---|---|---|
| | Example 4 | Example 5 | Example 6 | Example 7 |
| grade poloxamer | non-micronized | micronized | non-micronized | micronized |
| blending process | direct addition | direct addition | geometric dilution | geometric dilution |

The poloxamer in the drug product was quantified by HPLC-RI. The method was developed in accordance with Y. Mao et al., Quantitation of poloxamers in pharmaceutical formulations using size exclusion chromatography and colorimetric method, Journal of Pharmaceutical and Biomedical Analysis, 35 (2004) 1127-1142. Homogeneity of distribution was quantified based upon relative standard deviation (RSD) of samples and the results are compiled in the table here below:

| | comparative | | inventive | |
|---|---|---|---|---|
| | Example 4 | Example 5 | Example 6 | Example 7 |
| grade poloxamer | non-micronized | micronized | non-micronized | micronized |
| blending process | direct addition | direct addition | geometric dilution | geometric dilution |
| RSD | 12.6% | 11.1% | 8.6% | n.d. |

FIGS. 1 to 4 show photographs (10× magnitude) of pharmaceutical formulations of Examples 4-7. FIG. 1 shows comparative pharmaceutical formulation of Example 4 where the granules were blended with non-micronized poloxamer by direct addition. FIG. 2 shows comparative pharmaceutical formulation of Example 5 where the granules were blended with micronized poloxamer by direct addition. FIG. 3 shows inventive pharmaceutical formulation of Example 6 where the granules were blended with non-micronized poloxamer by geometric dilution. FIG. 4 shows inventive pharmaceutical formulation of Example 7 where the granules were blended with micronized poloxamer by geometric dilution.

As demonstrated by the above comparative data, blending the dry granules with poloxamer by geometric dilution has significant advantages that can be achieved with micronized poloxamer (Example 7) but also with non-micronized poloxamer (Example 6).

Pharmaceutical dosage forms made from the pharmaceutical compositions of Examples 4 and 7 as well as a comparative dosage form made by wet granulation where poloxamer is contain in the intragranular phase were tested in a clinical trial under fasting conditions. Coefficients of Variation (CV) of cₘₐₓ and of AUC_{0-∞} were determined and the in vivo data are compiled in the table here below:

| | extragranular surfactant | | |
|---|---|---|---|
| | (dry granulation) | | intragranular surfactant |
| | Example 4 | Example 7 | (wet granulation) |
| | comparative | inventive | comparative |
| CV %, cₘₐₓ | 52.8 | 40.1 | 36.4 |
| CV % AUC_{0-∞} | 58.1 | 46.6 | 44.7 |

As demonstrated by the above comparative data, based upon geometric dilution principle, pharmaceutical compositions can be prepared by an anhydrous granulation process (i.e. dry granulation) using surfactant such as poloxamer in the extragranular phase and nonetheless ensuring satisfactory and decent in vivo data with acceptable inter subject variability.

1-75. (canceled)

76. A pharmaceutical composition comprising:
(i) granules comprising Nilotinib or a physiologically acceptable salt and/or solvate thereof; and
(ii) an extragranular phase;
wherein the pharmaceutical composition comprises a surfactant which is contained in the extragranular phase, the granules, or both.

77. The pharmaceutical composition according to claim 1, wherein the surfactant is selected from the group consisting of
(a) poloxamers;
(b) ethoxylated fatty alcohols;
(c) alkyl phenol ethoxylates;
(d) polyoxyethylene fatty acid esters;
(e) polyoxyethylene esters of alpha-tocopheryl succinate;
(f) polyoxyethylene glycerol fatty acid esters;
(g) reaction products of a natural or hydrogenated castor oil and ethylene oxide;
(h) partial fatty acid esters of sorbitan;
(i) partial fatty acid esters of polyoxyethylene sorbitan;
(j) fatty acid esters or glycerol;
(k) polyglycolyzed glycerides;
(l) partial fatty acid esters of pentaerythritol;
(m) partial fatty acid esters of saccharose; and
(n) partial fatty acid esters of polyglycerol,
wherein the content of the surfactant is within the range of from 0.5 to 1.5 wt.-%, relative to the total weight of the composition.

78. The pharmaceutical composition according to claim 1, wherein the surfactant is contained entirely in the extragranular phase or a first fraction of the surfactant is contained in the granules and a second fraction of the surfactant is contained in the extrgranular phase,
wherein where the first fraction of the surfactant is contained in the granules, the relative weight ratio of the first fraction of the surfactant to the second fraction of the surfactant is within the range of from 90:1 to 1:90, or from 80:1 to 1:80, or from 70:1 to 1:70, or from 60:1 to 1:60, or from 50:1 to 1:50, or from 40:1 to 1:40, or from 30:1 to 1:30, or from 20:1 to 1:20, or from 10:1 to 1:10.

79. The pharmaceutical composition according to claim 1, wherein the surfactant has a HLB value within the range of 29±8 or the surfactant is micronized such that it has a weight average particle size as measured by sieve analysis in accordance with Ph. Eur. 2.9.38, of 50±40 μm; or has a particle size distribution as measured by sieve analysis in accordance with Ph. Eur. 2.9.38, wherein not more than 10 wt.-% retain on a sieve having a size of ≤106 μm, and not more than 50 wt.-% retain on a sieve having a size of ≤53 μm.

80. The pharmaceutical composition according to claim 1, wherein the Nilotinib is one or more of crystalline anhydrous Nilotinib hydrochloride salt and crystalline Nilotinib hydrochloride monohydrate salt and wherein the content of Nilotinib is within the range of 50±35 wt.-%, relative to the total weight of the composition and expressed as equivalent weight of Nilotinib in its non-salt non-solvate form.

81. The pharmaceutical composition according to claim 1, wherein the granules comprise a diluent selected from the group consisting of lactose, glucose, sucrose, sorbitol, mannitol, dextrates, dextrin, dextrose, microcrystalline cellulose, and powdered cellulose, and is within the range of 40±35 wt.-%, relative to the total weight of the composition.

82. The pharmaceutical composition according to claim 1, wherein the granules further comprise a disintegrant selected from the group consisting of cross-linked polyvinyl pyrrolidone, cross-linked sodium carboxymethylcellulose, cross-linked calcium carboxymethylcellulose, pregelatinized starch, clay, cellulose, soy polysaccharides, alginates, and gums,
wherein the disintegrant has a weight average particle size as measured by sieve analysis in accordance with Ph. Eur. 2.9.38, of 125±50 μm or the content of the disintegrant is within the range of 4.0±3.5 wt.-%, relative to the total weight of the composition.

83. The pharmaceutical composition according to claim 1, further comprising a glidant selected from the group consisting of colloidal silica, magnesium trisilicate, starch, talc, tribasic calcium phosphate, magnesium carbonate, magnesium oxide and powdered cellulose,
wherein the glidant is contained entirely in the extragranular phase or a first fraction of the glidant is contained in the granules and a second fraction of the glidant is contained in the extragranular phase and is within the range of 1.0±0.9 wt.-%, relative to the total weight of the composition.

84. The pharmaceutical composition according to claim 1, further comprising a lubricant selected from the group consisting of magnesium stearate, aluminum stearate, calcium stearate, polyethylene glycol, glyceryl behenate, stearic acid, hydrogenated castor oil, glyceryl monostearate and sodium stearyl fumarate,
wherein the lubricant is contained entirely in the extragranular phase or a first fraction of the lubricant is contained in the granules and a second fraction of the lubricant is contained in the extragranular phase and the content of the lubricant is within the range of 1.5±1.3 wt.-%, relative to the total weight of the composition.

85. The pharmaceutical composition according to claim 1, which excludes one or more of the following:
a binder selected from the group consisting of povidone, microcrystalline cellulose, hydroxyethylcellulose, hydroxypropylcellulose, and hydroxypropylmethylcellulose, citric acid or a salt thereof,
a diluent selected from the group consisting of lactose, glucose, sucrose, sorbitol, mannitol, dextrates, dextrin, dextrose, microcrystalline cellulose, and powdered cellulose,
an organic acid selected from acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, glutamic acid, aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid and ascorbic acid or a salt thereof.

86. The pharmaceutical composition according to claim 1, wherein
(i) the granules comprise one or more of the following:
Nilotinib or a physiologically acceptable salt and/or solvate thereof;
a diluent;
a disintegrant;
either all or a fraction of poloxamer
a fraction of glidant; and
a fraction of lubricant; and
(ii) the extragranular phase comprises one or more of the following:
either all or the remainder of the poloxamer;
the remainder of glidant;
the remainder of lubricant.

87. The pharmaceutical composition according to claim 1, wherein the surfactant is homogeneously distributed such that the relative standard deviation (RSD) of the surfactant content is between 8.8% and 10.9%.

88. The pharmaceutical composition according to claim 1, wherein the granules that are contained in the pharmaceutical composition have a size such that they pass a sieve having a mesh size of 1.0 to 2.0 mm but do not pass a sieve having a mesh size of between 0.5 mm an 0.8 mm.

89. A process for the preparation of a pharmaceutical composition according to claim 1 comprising the steps of:
(a) providing a formulation comprising:
Nilotinib or a physiologically acceptable salt and/or solvate thereof, and optionally one or more of the following:
a diluent,
a disintegrant,
a fraction of glidant,
a fraction of lubricant; and
a surfactant or a fraction of surfactant;
(b) dry granulating the formulation to obtain granules;
(c) optionally, sieving the granules such that during the step of sieving the granules removes large granules not passing a sieve of size 1.60 mm as well as small granules passing a sieve of size 0.80 mm;
(d) optionally, providing a surfactant formulation comprising the surfactant or the remainder of surfactant; and
(e) optionally, blending the surfactant formulation and the granules.

90. The process according to claim 14, wherein dry granulating is performed by roller compaction, wherein the roller compaction is performed at an average roller gap within the range of from 0.5 to 2.0 mm or at a pressure within the range of from 20 to 40 bar.

91. The process according to claim 14, wherein the process requires blending the surfactant formulation and the granules, wherein the step of blending comprises an initial sub-step in which the surfactant formulation is blended with a fraction of the granules, and wherein in subsequent sub-steps additional fractions of the granules are added to the blend.

92. The process according to claim 16, wherein in the step of blending the surfactant is blended with the granules until a homogeneous distribution is achieved such that the relative standard deviation (RSD) of the surfactant content is not more than between 8.8% and 10.9%.

93. The process according to claim 17, wherein the process comprises the additional steps of blending the granules obtained in step (b), the sieved granules obtained in step (c) or the product obtained in step (e)
(f) with the remainder of glidant; and optionally, sieving the thus obtained blend; and/or
(g) with the remainder of lubricant; and optionally, sieving the thus obtained blend.

94. A pharmaceutical composition that is obtainable by a process according to claim 16.

95. A pharmaceutical dosage form comprising a pharmaceutical composition according to claim 1, wherein the dosage form is one or more of the following: devoted for oral administration, is a capsule, such as a hard gelatin capsule, is for use in the treatment of cancer, including cancer that is chronic myeloid leukemia or a gastrointestinal stromal tumor, is administered orally either once daily or twice daily, provides an Coefficient of Variation (variability) of cₘₐₓ of not more than between 45% and 50%, and provides an AUC_{0-∞} of not more than between 50% and 55%.
